(19) United States
(12) Patent Application Publication
(10) Pub. No.: US 2018/0344944 A1
(43) Pub. Date: Dec. 6, 2018

(54) SYSTEM FOR CAP REMOVAL

(71) Applicant: Sanofi-Aventis Deutschland GMBH, Frankfurt am Main (DE)

(72) Inventors: Stefan Wendland, Frankfurt am Main (DE); Michael Harms, Frankfurt am Main (DE)

(21) Appl. No.: 15/778,265

(22) PCT Filed: Nov. 21, 2016
(86) PCT No.: PCT/EP2016/078255

§ 371 Date: May 23, 2018

(30) Foreign Application Priority Data
Nov. 27, 2015 (EP) 15196686.8

(51) Int. Cl. A61M 5/32

(52) U.S. Cl. CPC A61M 5/3204

(57) ABSTRACT

The present invention relates to an auto-injector device (10) for delivering a liquid medicament, the auto injector comprising an injector body, a syringe (18) received in the injector body, a needle (17) disposed in a first end of syringe to extend toward an opening of the injector body, a needle shield (25) removeably attached to the syringe to enclose the needle and an injector cap removeably received in the opening of the injector body to enclose the needle shield. The injector cap (12) comprises at least one engaging element to engage with the needle shield and the needle shield comprises a rupture portion. The at least one engaging element is configured to rupture the rupture portion as the cap is removed from the body. The at least one engaging element is configured to engage with at least a portion of the needle shield to retain at least a portion of the needle shield in the cap when the cap is removed from the body.

This application relates to an injector drug delivery device. Injector devices have application where regular injections by persons without formal medical training occur. This is common among patients where self-treatment enables effective management of their disease.

Such devices comprise a body containing a syringe and needle for dispensing a medicament. A cap attaches to the body to enclose the needle to both protect the needle from environmental damage and protect the user from injury by the needle. Generally, a needle shield is also provided as a closer fitting cover for the needle to prevent contamination of the needle. Conventionally, both the cap and the needle shield are removed separately to expose the needle prior to the injection.

It shall be appreciated that injector devices are often used by elderly or physically impaired patients that suffer limited dexterity and that such patients may experience difficulty removing the needle shield.

It is an object of the invention to address the problems mentioned above and provide an improved injector device.

According to the present invention there is provided an auto-injector device for delivering a liquid medicament, comprising an injector body, a syringe received in the injector body, a needle disposed in a first end of syringe to extend toward an opening of the injector body, a needle shield removeably attached to the syringe to enclose the needle, and an injector cap removeably received in the opening of the injector body to enclose the needle shield, wherein the injector cap comprises at least one engaging element to engage with the needle shield, and wherein the needle shield comprises a rupture portion, the at least one engaging element being configured to rupture the rupture portion as the cap is removed from the body, the at least one engaging element configured to engage with at least a portion of the needle shield to retain at least a portion of the needle shield in the cap when the cap is removed from the body.

Conventional injector devices require the user to remove the cap and the needle shield separately to expose the needle in order to make the device ready for injection. It shall be appreciated that infirm patients such as the elderly or physically impaired may find removing the needle shield more difficult than removing the cap due to the small size of the needle shield making it difficult to handle. According to the present invention the needle shield is removed in the same operation as removing the cap, thereby making the device easier for infirm patients to use.

The rupture portion may comprise a line of weakening.

Therefore the force required to remove the at least a portion of the needle shield is reduced to the benefit of infirm patients.

The line of weakening may be a notch that extends around an outer surface of the needle shield.

The notch provides an edge against which an engaging element can locate to separate the at least a portion of the needle shield along the line of weakening.

The at least one engaging element may comprise a pair of sprung arms depending from an internal surface of the cap and biased against the outer surface of the needle shield so that, as the cap is removed, an end of the sprung arms locates against an edge of the notch to react against said edge and cause the at least a portion of the needle shield to separate along the line of weakening.

The sprung arms bias against the side of the needle shield to so that as the cap is removed they are biased into the notch to locate against said edge of the notch.

The at least one engagement element may comprise a wall depending from an internal surface of the cap and extending into abutting relation with an edge of the notch so that, as the cap is removed, an end of the wall reacts against said edge to cause the at least a portion of the needle shield to separate along the line of weakening.

The engagement element may comprise a blade depending from an internal surface of the cap and extending toward the needle shield.

The rupture portion may comprise an end of the needle shield disposed in abutting relation with the first end of the syringe, the blade extending toward said end such that, when the cap is removed from the body, the blade makes an axial cut in said end so that said end is less stable.

With the needle shield less stable, less force is required to remove the needle shield to the benefit of infirm patients.

The needle shield may be made of an elastomeric material and wherein said end of the needle shield is retained on the first end of the syringe by elastic tension such that, when the cap is removed from the body, the resulting axial cut releases said elastic tension so that the needle shield is more easily removed from the first end of the syringe.

Therefore the force required to remove the at least a portion of the needle shield is reduced to the benefit of infirm patients.

The cap may comprise a stop depending from the internal surface of the cap, and the needle shield comprises an attached stop attached to a proximal end of the needle shield such that, when the cap is removed from the body, the stop and the attached stop abut so that the needle shield is retained in the cap to expose the needle.

Therefore the needle shield is removed from the syringe in the single step of removing the cap to the benefit of inform patients.

Also according to the invention there is provided a cap assembly for an auto injector device comprising a needle shield for removable attachment to a syringe of the auto injector device to enclose a needle thereof and an injector cap for removable attachment to a body of the auto injector device to enclose the needle shield. The injector cap comprises at least one engaging element to engage with the needle shield, and the needle shield comprises a rupture portion. The at least one engaging element is configured to rupture the rupture portion as the cap is removed from the body, the at least one engaging element is configured to engage with at least a portion of the needle shield to retain at least a portion of the needle shield in the cap when the cap is removed from the body.

According to the invention there is provided an auto injector device as described above comprising a cartridge of liquid medicament.

The terms “drug” or “medicament” which are used interchangeably herein, mean a pharmaceutical formulation that includes at least one pharmaceutically active compound.

The term “drug delivery device” shall be understood to encompass any type of device, system or apparatus designed to immediately dispense a drug to a human or non-human body (veterinary applications are clearly contemplated by the present disclosure). By “immediately dispense” is meant an absence of any necessary intermediate manipulation of the drug by a user between discharge of the drug from the drug delivery device and administration to the human or non-human body. Without limitation, typical examples of drug delivery devices may be found in injection devices, inhalers, and stomach tube feeding systems. Again without limitation, exemplary injection devices may include, e.g., syringes, autoinjectors, injection pen devices and spinal injection systems.

So that the present invention may be more fully understood embodiments thereof will now be described with reference to the accompanying drawings in which:

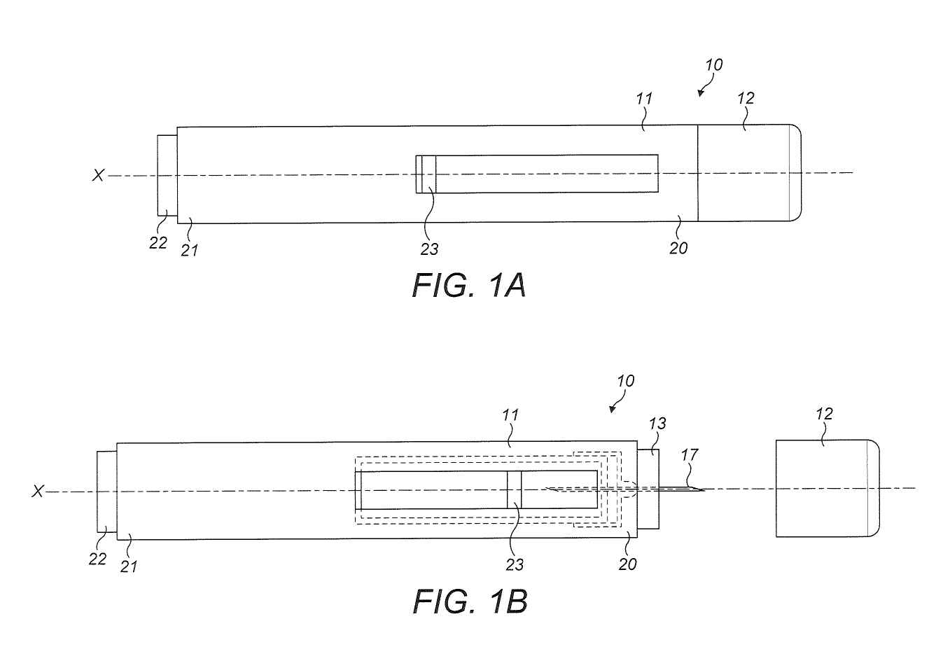

FIG. 1A shows an auto injector with a cap attached;

FIG. 1B shows the auto injector of FIG. 1A with the cap removed;

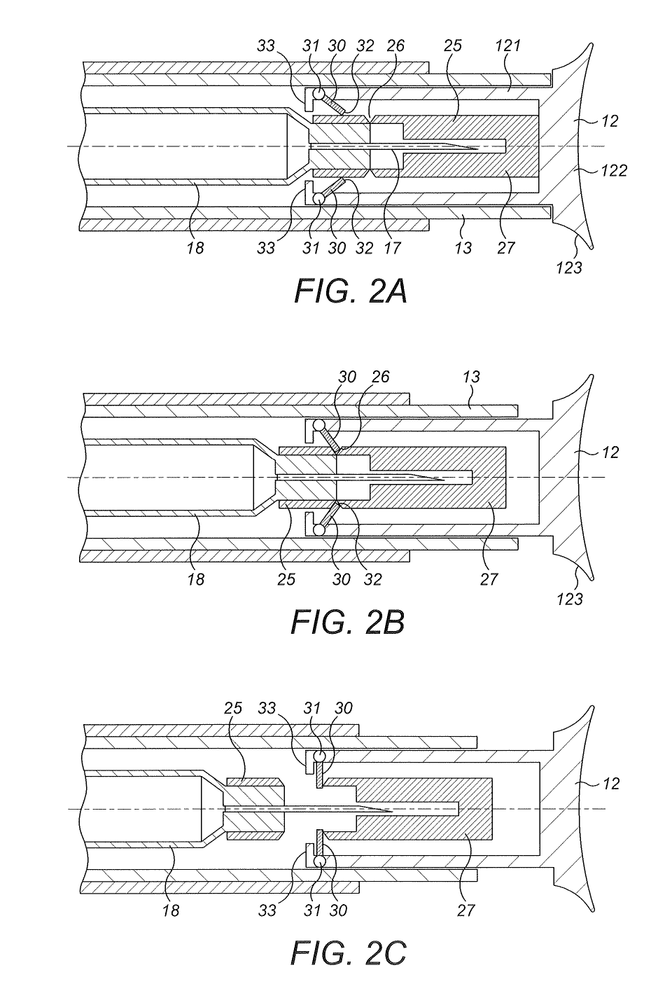

FIG. 2A shows a partial view of an auto injector with a cap attached according to an embodiment of the invention;

FIG. 2B shows a partial view of the auto injector of FIG. 2A with the cap partially removed to a first position;

FIG. 2C shows a partial view of the auto injector of FIG. 2A with the cap partially removed to a second position;

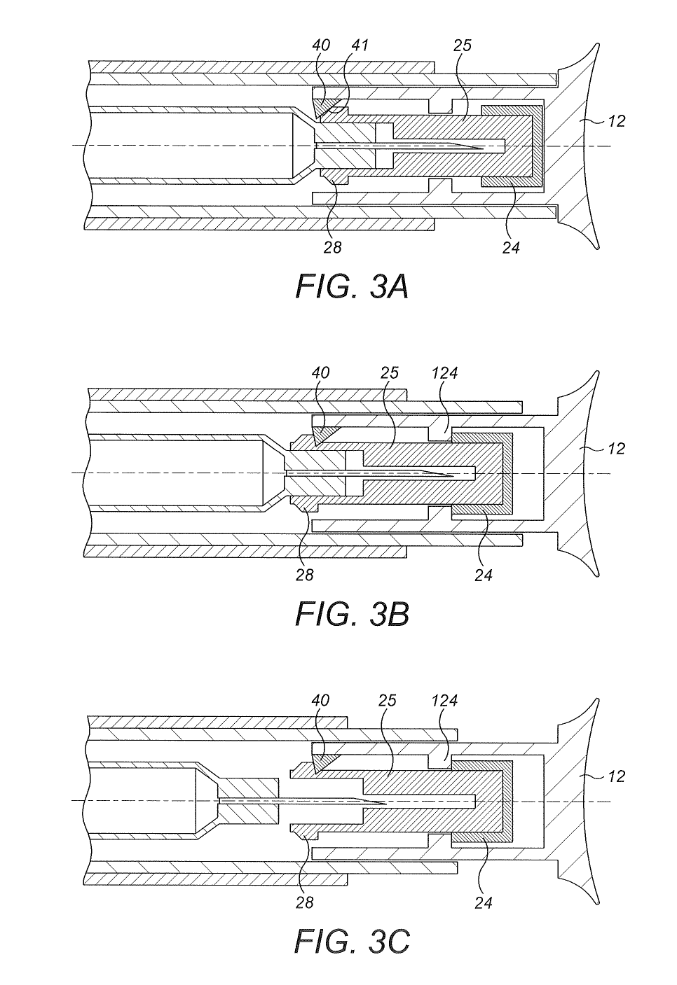

FIG. 3A shows a partial view of an auto injector with a cap attached according to another embodiment of the invention;

FIG. 3B shows a partial view of the auto injector of FIG. 3A with the cap partially removed to a first position;

FIG. 3C shows a partial view of the auto injector of FIG. 3A with the cap partially removed to a second position;

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 27 and 29 Gauge.

The delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device. The one or more automated functions of an auto-injector may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

According to some embodiments of the present disclosure, an exemplary drug delivery device 10 is shown in FIGS. 1A & 1B. Device 10, as described above, is configured to inject a medicament into a patient's body. Device 10 includes a housing 11 which typically contains a reservoir containing the medicament to be injected (e.g., a syringe) and the components required to facilitate one or more steps of the delivery process. Device 10 can also include a cap assembly 12 that can be detachably mounted to the housing 11. Typically a user must remove cap 12 from housing 11 before device 10 can be operated.

As shown, housing 11 is substantially cylindrical and has a substantially constant diameter along the longitudinal axis X. The housing 11 has a distal region 20 and a proximal region 21. The term “distal” refers to a location that is relatively closer to a site of injection, and the term “proximal” refers to a location that is relatively further away from the injection site. Device 10 can also include a needle sleeve 13 coupled to housing 11 to permit movement of sleeve 13 relative to housing 11. For example, sleeve 13 can move in a longitudinal direction parallel to longitudinal axis X. Specifically, movement of sleeve 13 in a proximal direction can permit a needle 17 to extend from distal region 20 of housing 11.

Insertion of needle 17 can occur via several mechanisms. For example, needle 17 may be fixedly located relative to housing 11 and initially be located within an extended needle sleeve 13. Proximal movement of sleeve 13 by placing a distal end of sleeve 13 against a patient's body and moving housing 11 in a distal direction will uncover the distal end of needle 17. Such relative movement allows the distal end of needle 17 to extend into the patient's body. Such insertion is termed “manual” insertion as needle 17 is manually inserted via the patient's manual movement of housing 11 relative to sleeve 13.

Another form of insertion is “automated,” whereby needle 17 moves relative to housing 11. Such insertion can be triggered by movement of sleeve 13 or by another form of activation, such as, for example, a button 22. As shown in FIGS. 1A & 1B, button 22 is located at a proximal end of housing 11. However, in other embodiments, button 22 could be located on a side of housing 11.

Other manual or automated features can include drug injection or needle retraction, or both. Injection is the process by which a bung or piston 23 is moved from a proximal location within a syringe (not shown) to a more distal location within the syringe in order to force a medicament from the syringe through needle 17. In some embodiments, a drive spring (not shown) is under compression before device 10 is activated. A proximal end of the drive spring can be fixed within proximal region 21 of housing 11, and a distal end of the drive spring can be configured to apply a compressive force to a proximal surface of piston 23. Following activation, at least part of the energy stored in the drive spring can be applied to the proximal surface of piston 23. This compressive force can act on piston 23 to move it in a distal direction. Such distal movement acts to compress the liquid medicament within the syringe, forcing it out of needle 17. Following injection, needle 17 can be retracted within sleeve 13 or housing 11. Retraction can occur when sleeve 13 moves distally as a user removes device 10 from a patient's body. This can occur as needle 17 remains fixedly located relative to housing 11. Once a distal end of sleeve 13 has moved past a distal end of needle 17, and needle 17 is covered, sleeve 13 can be locked. Such locking can include locking any proximal movement of sleeve 13 relative to housing 11.

Another form of needle retraction can occur if needle 17 is moved relative to housing 11. Such movement can occur if the syringe within housing 11 is moved in a proximal direction relative to housing 11. This proximal movement can be achieved by using a retraction spring (not shown), located in distal region 20. A compressed retraction spring, when activated, can supply sufficient force to the syringe to move it in a proximal direction. Following sufficient retraction, any relative movement between needle 17 and housing 11 can be locked with a locking mechanism. In addition, button 22 or other components of device 10 can be locked as required.

In FIGS. 2A and 3A an auto-injector is shown according to respective first and second illustrated embodiments of the invention. In FIGS. 2A and 3A said respective auto injectors are shown with the cap 12 attached. Common to both embodiments, a needle shield 25 is provided to enclose the needle 17. The needle shield 25 is an elongated tube with an open end in which the needle 17 is received. The open end of the needle shield 25 is received over a distal end of the syringe 18 so that an internal surface of the needle shield 25 tightly abuts an external surface of the syringe 18 to retain the needle shield 25 thereon.

The cap 12 is received in the sleeve 13 with an external surface of the cylindrical wall 121 of the cap 12 tightly abutting an internal surface of the sleeve 13 to retain the cap 12 thereon. With the cap 12 attached, an internal surface of the cylindrical wall 121 of the cap 12 faces, and is slightly spaced from, an external surface of the needle shield 25. Combined, the needle shield 25 and the cap 12 are referred to as the cap assembly.

The end wall 122 of the cap 12 has an extended portion 123 that extends outwardly beyond the perimeter of the sleeve 13 to provide a surface for the user of the auto-injector to pull against when removing the cap 12.

Before the injection can occur, both the cap 12 and at least a portion of the needle shield 25 must be removed from the device to expose the needle 17. According to the invention, the cap 12 comprises an engaging element to engage a rupture portion of the needle shield 25 so that, as the cap 12 is removed, the engaging element causes the rupture portion to rupture and detach at least a portion of the needle shield 25 from the syringe 18. The at least a portion of the needle shield is retained in the cap as it is removed to expose the needle 17.

In the first illustrated embodiment of the invention, shown in FIGS. 2A to 2C, the engaging element comprises a pair of elongate arms 30 that depend from the internal surface of the cylindrical wall 121 of the cap 12 to extend obliquely away from the internal surface into abutting relation with the external surface of the needle shield 25. The arms 30 are mounted to the internal surface by a torsion spring 31 which biases a tip 32 of the arm 30 up against the needle shield 25.

In this embodiment the rupture portion of the needle shield 25 comprises a notch 26 formed around the circumference of the outer surface of the needle shield 25. The notch 26 serves as a line of weakening that enables a portion 27 of the needle shield 25 enclosing the needle to be separated from the syringe 18 when the cap 12 is removed. Said portion 27 of the needle shield 25 that is separated from the syringe 18 is herein referred to as the separable portion 27. The separable portion 27 is retained in the cap 12 as the cap 12 is removed to expose the needle 17.

To remove the cap 12, the user pulls against the extended portion 123 of the cap 12, in doing so an axial force is applied to the cap 12 to displace the cap 12 distally away from the sleeve 13. As the cap 12 is removed, the tips 32 of the arms 30 move into an engaging position to engage with a distal edge of the notch 26 as shown in FIG. 2B. The axial force applied to the cap 12 is transferred through the arms 30 and into said edge of the notch 26 to react against the separable portion 27 of the needle shield 25 and to cause the line of weakening to rupture and the separable portion 27 of the needle shield 25 to separate from the syringe 18 along the line of weakening.

In one example of the first embodiment the tips 32 of the arms 30 are disposed opposite each other so that, as the cap 12 is removed, the notch 26 in the needle shield 25 is pinched between the opposing tips 32 of the arms 30. It shall be appreciated that this pinching action increases local stress in the line of weakening and encourages separation of the separable portion 27 of the needle shield 25 from the syringe 18.

With the separable portion 27 of the needle shield 25 separated from the syringe 18 as shown in FIG. 2C, the arms 30 rotate under the action of the torsion spring 31 into a closed position to abut a respective stop 33. With the arms 30 disposed in the closed position the arms 30 are arranged perpendicular to the internal surface of the cap 12 such that they partially block the opening in the cap 12. The arms 30 extend across the opening in the cap 12 to the extent that the separable portion 27 of the needle shield 25 is prevented from passing through the opening irrespective of the orientation of the cap 12.

Although in the first embodiment as described above a single pair of elongate arms 30 is provided, it shall be appreciated that in other examples of this embodiment more than two arms 30 may be provided.

Although in the first embodiment as described above the arms 30 are mounted by a torsion spring 31, in another unillustrated example of this embodiment the arms are mounted directly to the internal surface of the cap 12. For example, the arms may be formed integrally with the cap 12. In such examples, the arms are resiliently deformable and biased against the needle shield 25. As the cap 12 is removed, tips of the resilient arms move into an engaging position to engage with the distal edge of the notch 26 to separate the separable portion 27. With the separable portion 27 of the needle shield 25 separated from the syringe 18, the resilient arms move into a closed position in which the resilient arms are arranged so as to partially block the opening in the cap 12. The resilient arms extend across the opening in the cap 12 to the extent that the separable portion 27 of the needle shield 25 is prevented from passing through the opening irrespective of the orientation of the cap 12.

In another (unillustrated) embodiment, the engaging element comprises a wall depending from the internal surface of the cap 12. The wall may be resiliently deformable and extend circumferentially around in the internal surface of the cap. With the cap 12 attached to the auto-injector as described above, the wall extends into abutting relation with the distal edge of the notch 26. Therefore, as the cap 12 is removed, the axial force applied to the cap 12 is transferred through the wall and into said edge of the notch 26 to react against the separable portion 27 of the needle shield 25 and to cause the separable portion 27 of the needle shield 25 to separate from the syringe 18 along the line of weakening. The wall partially blocks the opening in the cap 12 so to prevent the separable portion 27 of the needle shield 25 from passing through the opening irrespective of the orientation of the cap 12.

According to yet another (unillustrated) embodiment, the engaging element comprises a first stop depending from an external surface of the separable portion 27 of the needle shield 25 and a second stop depending from the internal surface of the cap 12, the first and second stops have respective facing surfaces. The second stop is disposed closer to the proximal region 21 than the first stop, so that as the cap 12 is removed, respective facing surfaces of the stop abut to transfer the axial force applied to the cap 12 to the separable portion 27 of the needle shield 25 to cause the separable portion 27 of the needle shield 25 to separate from the syringe 18 along the line of weakening.

It shall be appreciated that in such an embodiment the interface between the first stop and the second stop prevents the separable portion 27 of the needle shield 25 from passing through the opening of the cap 12 irrespective of the orientation of the cap 12.

Referring now to the second illustrated embodiment shown in FIGS. 3A to 3C, the engaging element comprises a blade 40 depending from the internal surface of the cap 12. The blade 40 has a leading edge 41 configured to make an axial cut in the rupture portion of the needle shield 25 as the cap 12 is removed. In this embodiment the rupture portion comprises the portion of the needle shield 25 disposed over the distal end of the syringe 18. The leading edge 41 of the blade 40 is disposed adjacent the rupture portion so that, as the cap 12 is removed, the leading edge 41 of the blade 40 makes an axial cut in the rupture portion. Therefore the cut extends through the external surface of the needle shield 25 where the needle shield 20 abuts the distal end of the syringe 18. The cut reduces the stability of the portion of the needle shield 25 in contact with the proximal end of the syringe 18 so that it is easier to remove. In other words, with the needle shield 25 in the less stable state, the axial force required to remove the needle shield 25 is reduced.

In one example of this embodiment, the needle shield 25 is made of an elastomeric material and the needle shield 25 is held in tightly abutting relation with the distal end of the syringe 18 by elastic tension. In such embodiments the rupture portion may be a region of increased wall thickness 28 where the needle shield 25 abuts the syringe 18. As the cap 12 is removed, the leading edge 41 of the blade 40 makes an axial cut in region of increased wall thickness 28, as shown in FIG. 3B. The axial cut causes partial release of the elastic tension so that the needle shield 25 only lightly abuts the syringe 18, thus reducing the axial force required to remove the needle shield 25 from the syringe 18.

According to the second illustrated embodiment, a further engaging element is provided comprising a stop 124 depending from the internal surface of the cap 12, an attached stop 24 is also provided depending from the external surface of the needle shield 25. The stop 124 is located closer to the proximal region 21 than the attached stop 24 so that, as the cap 12 is removed, the stop 124 and the attached stop 24 abut to transfer the axial force applied to the cap 12 to the needle shield 25 to cause the needle shield 25 to be removed from the syringe 18 with the cap 12, as shown in FIG. 3C. Therefore, the entire cap assembly is removed. The stop 124 and the attached stop 24 are spaced apart axially a distance at least equivalent to the width of the region of increased wall thickness 28 so that the needle shield 25 is less stable before it is removed from the syringe 18, therefore reducing the axial force required to remove the needle shield 25.

It shall be appreciated that the interface between the stop 24 and the attached stop 124 prevents the needle shield 25 from passing through the opening of the cap 12 irrespective of the orientation of the cap 12 so that when the cap 12 is removed the needle shield 25 is retained in the cap 12 to expose the needle 17.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The term “drug delivery device” shall encompass any type of device or system configured to dispense a drug or medicament into a human or animal body. Without limitation, a drug delivery device may be an injection device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), an implantable device (e.g., drug- or API-coated stent, capsule), or a feeding system for the gastro-intestinal tract. The presently described drugs may be particularly useful with injection devices that include a needle, e.g., a hypodermic needle for example having a Gauge number of 24 or higher.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders.

Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15^{th} edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refer to any substance which is sufficiently structurally similar to the original substance so as to have substantially similar functionality or activity (e.g., therapeutic effectiveness). In particular, the term “analogue” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N- tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®, Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide / HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten. An example of an oligonucleotide is, for example: mipomersen sodium (Kynamro®), a cholesterol-reducing antisense therapeutic for the treatment of familial hypercholesterolemia. Examples of DPP4 inhibitors are Vildagliptin, Sitagliptin, Denagliptin, Saxagliptin, Berberine. Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)₂ fragments, which retain the ability to bind antigens. The antibody can be polyclonal, monoclonal, recombinant, chimeric, de-immunized or humanized, fully human, non-human, (e.g., murine), or single chain antibody. In some embodiments, the antibody has effector function and can fix a complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

1. An auto-injector device for delivering a liquid medicament, comprising:
an injector body,
a syringe received in the injector body,
a needle disposed in a first end of syringe to extend toward an opening of the injector body,
a needle shield removeably attached to the syringe to enclose the needle, and
an injector cap removeably received in the opening of the injector body to enclose the needle shield, wherein the injector cap comprises at least one engaging element to engage with the needle shield, and wherein the needle shield comprises a rupture portion, the at least one engaging element being configured to rupture the rupture portion as the cap is removed from the body, the at least one engaging element configured to engage with at least a portion of the needle shield to retain at least a portion of the needle shield in the cap when the cap is removed from the body.

2. An auto injector device according to claim 1, wherein the rupture portion comprises a line of weakening.

3. An auto injector device according to claim 2, wherein the line of weakening is a notch that extends around an outer surface of the needle shield.

4. An auto injector device according to claim 3, wherein the at least one engaging element comprises a pair of sprung arms depending from an internal surface of the cap and biased against the outer surface of the needle shield so that, as the cap is removed, an end of the sprung arms locates against an edge of the notch to react against said edge and cause the at least a portion of the needle shield to separate along the line of weakening.

5. An auto injector device according to claim 3, wherein the at least one engagement element comprises a wall depending from an internal surface of the cap and extending into abutting relation with an edge of the notch so that, as the cap is removed, an end of the wall reacts against said edge to cause the at least a portion of the needle shield to separate along the line of weakening.

6. An auto injector device according to claim 1, wherein the at least one engaging element comprises a blade depending from an internal surface of the cap and extending toward the needle shield.

7. An auto injector device according to claim 6, wherein the rupture portion is an end of the needle shield disposed in abutting relation with the first end of the syringe, the blade extending toward said end such that, when the cap is removed from the body, the blade makes an axial cut in said end so that said end is less stable.

8. An auto injector device according to claim 7, wherein the needle shield is made of an elastomeric material and wherein the end of the needle shield is retained on the first end of the syringe by elastic tension such that, when the cap is removed from the body, the resulting axial cut releases said elastic tension so that the needle shield is more easily removed from the first end of the syringe.

9. An auto injector device according to claim 8, wherein the at least one engaging element comprises a stop depending from the internal surface of the cap, and the needle shield comprises an attached stop attached to a proximal end of the needle shield such that, when the cap is removed from the body, the stop and the attached stop abut so that the needle shield is retained in the cap to expose the needle.

10. A cap assembly for an auto injector device comprising:
a needle shield for removable attachment to a syringe of the auto injector device to enclose a needle thereof,
an injector cap for removable attachment to a body of the auto injector device to enclose the needle shield, wherein the injector cap comprises at least one engaging element to engage with the needle shield, and wherein the needle shield comprises a rupture portion, the at least one engaging element being configured to rupture the rupture portion as the cap is removed from the body, the at least one engaging element configured to engage with at least a portion of the needle shield to retain at least a portion of the needle shield in the cap when the cap is removed from the body.

11. An auto injector device according to any of claims 1 to 9 comprising a cartridge of liquid medicament.
